(19) United States
(12) Patent Application Publication
(10) Pub. No.: US 2011/0118360 A1
(43) Pub. Date: May 19, 2011

(54) AGENT FOR ACTIVATING STEM CELLS

(76) Inventors: Vladislav Nikolaevich Laskavy, Saratov (RU); Dmitriy Vladimirovich Goryunov, Zurich (CH)

(21) Appl. No.: 12/737,462

(22) PCT Filed: Mar. 30, 2009
(86) PCT No.: PCT/RU2009/000148

§ 371 Date: Jan. 14, 2011

(30) Foreign Application Priority Data
Jul. 17, 2008 (RU) 2008129554

(51) Int. Cl. A61K 31/115

(52) U.S. Cl. 514/694

(57) ABSTRACT

The invention relates to medicine, in particular to medicinal preparations directed at activating own stem cells of a human body. The aim of the invention is to develop a non-toxic agent which does not produce side effects and is used for activating the stem cells of an organism. The proposed method for producing the agent involves taking a 37-40% medicinal formaldehyde solution and adding it into a 0.9-0.95% sterile sodium chloride solution used for injections in such a way that a 0.00003-0.003% formaldehyde solution is obtained. The agent should be stored in a dark place at a temperature of 15-35° C.

### CROSS-REFERENCE TO RELATED APPLICATIONS

This application is a U.S. national stage application of a PCT application PCT/RU2009/000148 filed on 30 Mar. 2009, published as WO/2010/008317, whose disclosure is incorporated herein in its entirety by reference, which PCT application claims priority of a Russian Federation application RU2008129554 filed on 17 Jul. 2008.

### FIELD OF THE INVENTION

The present invention relates to medicine, in particular, to drugs designed to activate own stem cells of a human.

### BACKGROUND OF THE INVENTION

Human stem cells are used in modern medicine in treating many diseases, including cancer, cardiology and others. Stem cells can be derived from bone marrow and from umbilical cord blood and peripheral blood, as well as from embryonic tissues.

Stem cells are divided into mesenchymal (they are able to differentiate into cells of tissues of mesodermal origin), hematopoietic (precursors of blood cells), neuronal (progenitor cells of the nervous system), and others. Mesenchymal stem cells (MSCs) are often used as a graft, because they will enhance their own body reserves, promote the formation of cytokines and growth factors. MSCs are precursors of osteoblasts and promote the formation of remodeling units.

In the practice of cell therapy there are widely distributed and used means of activation of stem cells, based on bringing in new stem cells into the organism, rather than activation of its own stem cells.

In particular, there is known a means to activate the stem cells through glycoprotein (Russian Federation Patent No. 2,272,810, published 27 Mar. 2006), which contains glycoprotein polyclonal immunoglobulins of IgG class with an average molecular weight of 150 kDa, obtained as a humoral immune response to complex antigens, CD34+ stem cells. This preparation operates on the full range of antigenic determinants on the surface and inside of stem cells, which leads to their activation. However, the preparation is activated with their own stem cells. The process of obtaining the drug is durable and requires great effort.

There is known a means to activate the stem cells (drug Filgastrim (Neupogen) of F. Hoffman-la Roshe), being a glycoprotein consisting of 175 amino acids and having a molecular weight of 187-189 kilodaltons (kDa) obtained by expression of the human gene 5gdl-g31 into the E. coli genome. The means contains an additional amino acid—methionine. Filgastrim shortens the maturation period of neutrophils from the progenitor cells from 5 to 1 day, speeds up the output of mature neutrophils from the bone marrow into the blood (B. Lord et al. Proc. Of the National Academy of Sci. Of USA, 1992, 1986, p. 9499 -9503), enhances the chemotaxis of neutrophils (SPColgan et al., Experim. Hematology, 1992, 1920, p. 1229-1234). It is used for standard cytotoxic chemotherapy, bone marrow transplantation, to mobilize the natural colony-stimulating factor in chronic neutropenia, to accelerate the healing of wounds, etc.

However, the application of Filgastrim causes complications: bone pain, enlarged spleen, skin reactions, etc. Furthermore, the cost of Filgastrim treatment is sufficiently high and constitutes about U.S. $1500-3000.

The closest to the claimed drug is considered a drug called “bortesomib” (see www.rlsnet.ru/mnn_bortezomib.html), which is a modified boric acid being a highly selective reversible inhibitor of 26S proteasome activity, which is present in the nucleus and in the cytosol of all eukaryotic cells, and which catalyzes the splitting of main proteins involved in the life cycle of cells. The chemical name of the drug is [(1R)-3-methyl-1 [[(2S)-1-oxo-3-phenyl-2-[(pirasinilcarbonil)amino]propyl]amino]butyl], its brutto-formula is: C₁₉H₂₅BN₄O₄.

In vivo, bortesomib causes a slowdown in many experimental models of human tumors, including multiple myeloma. The drug is diluted with 0.9% NaCl to a concentration of 1 mg/ml, is administered intravenously. The main purpose of the drug is the treatment of multiple myeloma.

It was also established (sm.www.sciencedaily.com/releases/2008/01/080124 173,809. Htm), that bortesomib reduces the destruction of bone tissue, increases the activity of osteoblasts and bone formation, has a direct pharmacological effect on mesenchymal stem cells. Thus, the drug activates stem cells in the body, reinforcing its reparative ability, in contrast to the cell therapy based on bringing new stem cells into the organism, which cell therapy is widespread and widely used in practice.

The main drawback of this drug is its high toxicity and many side effects: on the part of blood (thrombocytopenia, anemia), with the digestive system (nausea, vomiting, ileus, acute pancreatitis, hepatitis), with the nervous system (headache, loss of consciousness, dysfunction of autonomic nervous system, convulsions), with Parties, with the cardiovascular system (drop in blood pressure, congestive heart failure), etc.

### AIM AND BRIEF DESCRIPTION OF THE INVENTION

The invention aims at solving the task by creating an agent (preparation) for activating the body's own stem cells, which agent is non-toxic and free of known side effects.

To solve this problem, the agent for activation of stem cells consists of an active ingredient of chemical origin and sodium chloride for injections. According to the invention, the active ingredient of chemical origin is provided in the form of formaldehyde in the amount of 0.00003-0.004 (wt. %); and the rest weight amount of the agent constitutes sodium chloride for injections of a 0.85-0.95% concentration.

The sources of patent and scientific information, known to the authors (i.e. inventors of the present invention), do not describe any means for the activation of stem cells, which means being non-toxic, having no side-effects, being based not on infusing stem cells into the body, but would be based on the action of the own cells of the body, namely, on spleen cells and red bone marrow.

There is known an immunomodulatory agent containing formaldehyde and sodium chloride (see Russian Federation Patent No. 2077882, published 27 Apr. 1997), wherein the concentration of formaldehyde is 0.07-0.24 wt. %. The mentioned patent, however, does not suggest or motivate any changes of the concentration of formaldehyde beyond the range of 0.07-0.24 wt. %.

However, the aforementioned drug does not activate the stem cells, but only has immunomodulatory effects. The present inventors were first, who identified the action of the drug in the concentration of formaldehyde within the range of 0.00003-0.004 wt. % that increases the aerobic oxidation, which in turn limits its effect on the normal cells and leads to the death of cancer cells. Thus, the concentration of formaldehyde in the range of 0.00003-0.004 wt. % produces a new and unexpected result stated above. Therefore the concentration range from 0.00003 to 0.004 wt. % is critically important for carrying out the present invention and makes it unobvious to those skilled in the art.

An aqueous solution of formaldehyde is a transparent colorless liquid with a peculiar pungent smell, capable of mixing with water and alcohol in any proportions.

Formaldehyde is a representative of the class of aldehydes HCOH. It is a colorless gas with a pungent smell, having a molecular mass of 30.03; its density at 20° C. is 0.815, the melting point is 92° C., the boiling temperature is 19.2° C. It is soluble in water and alcohol. It is easy to polymerize, forming paraformaldehyde upon the polymerization in aqueous medium, and forming polyoxymetilene (POM) in and environments (butane, hexane).

An isotonic sodium chloride solution for injections is a colorless transparent liquid of a salty taste. The solution was sterile, pyrogen-free. Sodium chloride typically appears in the form of cubic crystals or white crystalline powder; it has a salty taste, and has no smell. It is soluble in water (1:3).

### DETAIL DESCRIPTION OF PREFERRED EMBODIMENTS OF THE INVENTION

While the invention may be susceptible to embodiment in different forms, there are described in detail herein, specific embodiments of the present invention, with the understanding that the present disclosure is to be considered an exemplification of the principles of the invention, and is not intended to limit the invention to that as described herein.

The claimed agent is a clear, colorless, odorless liquid; it has a slightly salty taste. The claimed agent is prepared as follows.

One should: (a) take a 37-40% medical solution of formaldehyde, and (b) add it to a sterile 0.9-0.95% solution of sodium chloride for injections to obtain the requisite 0.00003-0.003 wt % solution of formaldehyde. The so obtained solution is kept in a dark place at a temperature of 15-35° C.

### Example 1

Take 0.01 ml of 37% medical solution of formaldehyde, add it into 99.99 ml of a sterile 0.9% (or 0.95%) isotonic sodium chloride solution. The mixture of solutions is thoroughly intermingled. The final concentration of formaldehyde in the so obtained agent will be 0.0037 wt. %.

### Example 2

Take 0.0001 ml of 37% medical solution of formaldehyde, add it to 99.9999 ml of a sterile 0.9% (or 0.95%) isotonic sodium chloride solution. The mixture of solutions is thoroughly intermingled. The final concentration of formaldehyde in the so obtained agent will be equal to 0.000037 wt. %.

### Example 3

Take 0.01 ml of a 40% medicinal solution of formaldehyde. The agent is prepared in accordance with the above instruction of Example 2. The final concentration of formaldehyde will be equal to 0.004 wt. %.

### Example 4

Take 0.0001 ml of a 40% medical solution of formaldehyde; add it to 99, 9999 ml of a sterile 0.95% isotonic sodium chloride solution. The mixture of solutions is thoroughly intermingled. The final concentration of formaldehyde in the so obtained agent will be equal to 0.00003 wt. %.

To prove the activation of stem cells, a study was carried out as to the proliferative action of the inventive drug, and as to its possible toxic action on the spleen cells and bone marrow.

The experiments used 3-month-old mice of line C57B1/6 (males). The mice were decapitated and the spleen and red bone marrow were extracted under sterile conditions. Fragments of the spleen were homogenized thoroughly in a glass homogenizer in medium 199, filtered through a sterile gauze and washed 3 times in medium 199 for 5 minutes at 1500 rpm.

Bone marrow cells were extracted with a syringe and medium 199 from the femur. They were then carefully pipetted and washed with the above method.

After this, splenocytes and bone marrow cariotsites were transferred into a full culture medium (medium RPMJ-1640+10% FCS (fetal calf serum)+2 mM L-glutamine+25 mM Hepes-buffer+2+mercaptoethanol 4 g/ml gentamicin) and adjusted to a concentration of 10⁶ of nucleated cells per 1 mL. After the above mentioned procedures, the number of viable cells was 93-98%.

Cell culturing was performed under sterile conditions in a humid atmosphere with 5% CO₂. The number of live and dead cells was evaluated using a luminescence microscope after the adding to the cell suspension mixture of solutions of acridine orange (0.25 mg/ml) and ethidium bromide (0.25 mg/ml). The proliferative activity of cells was assessed after adding to them 1 mkKYu 3H-thymidine (specific activity) for four hours after the incubation.

After the incubation with thymidine, the cells were transferred onto glass-fiber filters, washed with a large excess of water, fixed by a 96° alcohol and then dried in air. Thereafter, the filters were immersed in standard toluene scintillator-based POP and POPOP (3 samples per one dilution of the drug). Radioactivity of the samples was calculated using a β-gauge (the number of β-decay pulses of ³H-thymidine per 1 minute).

In the process of cell cultivation, the drug was added at different concentrations, and the percentage of dead cells after a 24 hours incubation was taken into account.

The results of determining the toxicity of the inventive agent in different concentrations are presented in Table 1 below.

TABLE 1
The percentage of dead cells (splenocytes and bone marrow cells) 3-month-old
mice S57VL/6 after 24 h incubation with different concentrations of the drug.

| | Drug concentration | | | | | | | |
|---|---|---|---|---|---|---|---|---|
| Cells | 0.0035 | 0.00175 | 0.000875 | 0.00044 | 0.00011 | 0.000055 | 0.0000273 | Control |
| Splenocytes | 65 | 65 | 65 | 73 | — | 76 | 45 | 31 |
| Cells bone | 51 | 66 | 57 | 64 | 69 | 56 | — | 33 |
| brain | | | | | | | | |

From Table 1 that different concentrations of the drug (0,0035-0,0000273) as when added to splenocytes and cariotsytes, increase the number of dead cells compared with control in 1.4-2.5 times.

The result of studying the proliferative capacity of splenocytes and cariotsytes are presented in Table 2.

TABLE 2
Proliferation of splenocytes and bone marrow cells after 24
h incubation with different concentrations of the drug.

| | Drug concentration | | | | | | | |
|---|---|---|---|---|---|---|---|---|
| Cells | 0.0035 | 0.00175 | 0.000875 | 0.00044 | 0.00011 | 0.000055 | 0.0000273 | Control |
| Splenocytes | 451 | 770 | 1750 | 2439 | — | 5050 | 4000 | 1200 |
| Cells bone | 6000 | 9000 | 4400 | 3300 | 1500 | 700 | — | 711 |
| brain | | | | | | | | |

Table 2 shows that the proliferation of splenocytes compared to the control numbers increases in 1.5-4.2 times, and the number of kariotsitov increases from 2.1 to 12.7 times.

We studied the proliferative activity of splenocytes in a comparative aspect after 24 and 44 hours of incubation. In this case, the proliferation activity was estimated by the number of pulses per minute of ³H-thymidine injected into the cell culture for 4 hours. The results are presented in Table 3.

TABLE 3
Proliferative activity of splenocytes 100,000 3-month mouse S57VL/6 after
24 and 44 h incubation with different concentrations of the drug.

| Term | Drug concentration | | | | | | | |
|---|---|---|---|---|---|---|---|---|
| incubation | 0.0035 | 0.00175 | 0.000875 | 0.00044 | 0.00011 | 0.000055 | 0.0000273 | Control |
| 24 hours | 451 | 770 | 1750 | 2439 | — | 5050 | 4000 | 1200 |
| 44 hours | 2692 | 6229 | 9964 | 25528 | 13247 | 12756 | 9930 | 1826 |

Table 3 shows that at all concentrations of the drug, the cell proliferation increases with the incubation period compared with the control numbers in at 1.5-14 times.

These results indicate that the inventive drug in the in vitro conditions enhances the proliferation of spleen cells and bone marrow. In this case, the partial cell death is not determinative in the process of reproduction thereof, and it does not affect the enhancement of proliferation of the remaining normal cells.

The inventive drug has been tried out in the working concentration on a limited number of patients having non-healing trophic ulcers, postoperative fistula, and fracture. The drug was injected intramuscularly 1 time per week during 1-2 months.

### Example I

Patient N., aged 35, with postoperative fistula. Following a course of treatment for 2 months there was a full healing of wounds without re-operative intervention.

### Example II

Patient S., 72 years of age, had incurable trophic ulcer. After the course of treatment by the drug within 1 month, there occurred a purification and complete healing of the ulcer.

### Example III

Patient K., aged 45, had fracture of the ankle joint. After the treatment by the drug for 1.5 months, there was a restoration of bone tissue without any complications. The acceleration of recovery process was radiologically proven.

Thus, the claimed agent (drug) applied in vitro to a limited number of patients has demonstrated the possibility of activation of own stem cells of the human body.

1. An agent for activating own stem cells of an organism, said agent having a total weight and said agent consisting of:
an active ingredient of chemical origin provided in the form of formaldehyde in any weight percentage selected from the range of 0.00003-0.004 wt. % of the total weight; and
sodium chloride in the concentration selected from the range of 0.85-0.95%, wherein the weight of said sodium chloride constitutes the remaining portion of the total weight of said agent.
